(19) United States
(12) Patent Application Publication
(10) Pub. No.: US 2008/0172045 A1
(43) Pub. Date: Jul. 17, 2008

(54) Acne treatment device

(76) Inventors: Steven C. Shanks, Mckinney, TX (US); Ryan Maloney, Sun City, AZ (US)

Correspondence Address: ETHERTON LAW GROUP, LLC, 5555 E. VAN BUREN STREET, SUITE 100, PHOENIX, AZ, 85008, US

(21) Appl. No.: 12/009,340

(22) Filed: Jan. 18, 2008

Related U.S. Application Data
(63) Continuation-in-part of application No. 10/973,608, filed on Oct. 25, 2004, now abandoned.
(60) Provisional application No. 60/514,162, filed on Oct. 24, 2003.

(51) Int. Cl. A61B 18/18; A61B 18/08

(52) U.S. Cl. 606/3; 606/29

(57) ABSTRACT

A handheld probe treats acne and other skin conditions using a combination of optical and thermal therapy. The device contains light emitting devices capable of impinging a treatment area with laser light. In the preferred embodiment, the light emitting devices emit a combination of red and blue light to treat acne causing bacteria and prevent inflammation. The emitted light can be formed into therapeutically effective shapes by one or more optical arrangements. The device also contains a thermally conductive plate heated by a heater. The thermally conductive plate contacts the skin and warms it, enlarging the pores in the treatment area. One or more contact switches activate the device when brought into or near contact with the treatment area. In the preferred embodiment, the device is powered by a rechargeable battery.

### CROSS-REFERENCE TO RELATED APPLICATIONS

This application claims the benefit of co-pending provisional application No. 60/881,290, filed Jan. 18, 2007, and is a continuation-in-part of utility patent application Ser. No. 10/973,608, filed Oct. 25, 2004, and claiming the benefit of provisional application No. 60/514,162, filed October 24, 2003.

### FIELD OF INVENTION

This invention relates to a device for treating inflammatory and non-inflammatory acne. In particular, it relates to a contact device utilizing thermal and optical treatment.

### BACKGROUND

Acne is a chronic disease of the pilosebaceous follicle, affecting up to 80% of individuals at some stage of their life. It is particularly prevalent amongst adolescents. Acne can not only cause skin disfigurement and scarring, but can have a harmful effect on an individual's psychological development, resulting in emotional scarring, which may lead to social phobias, clinical depression and even suicide. It is therefore desirable to have an effective treatment for the acne.

Most modern acne treatments are focussed towards “opening” pores as well as killing propionibacterium acnes. The most common form of acne treatment is topical application of washes and creams. While these are heavily promoted by pharmacology companies they only provide modest effectiveness. The treatments are expensive, inconvenient and often have side-effects. Many people, especially adolescents stop the treatment due to the inconvenience and thereby exacerbate their problem.

It has recently become known that non-invasive light treatments can control or reduce acne. Propionibacterium acnes produce and accumulate porpyhrins, specifically endogenous porphyrins such as coproporphyryin-photosensitizers, which can absorb optical energy in the blue/violet part of the spectrum. Blue light has a limited depth of penetration and therefore may not be effective in treating propionibacterium acnes in all cases. Red light has a greater penetration depth but is less effective in treating propionibacterium acnes. However, red light may have other beneficial effects, such as stimulating cytokine release that has anti-inflammatory properties.

Several known devices treat various dermatological conditions, including acne, by treating affected skin with non-invasive light. These devices use various combinations of laser light sources and light emitting diodes functioning at multiple wavelengths, including blue, red, and green light. Particular combinations of treatment parameters, including pulse rate, peak power, and energy density, are disclosed for treating cell mediated inflammations of the skin and viscera. The devices may be handheld and the effect of the light treatments may be enhanced with topical compositions. However, none of these devices recognize the important role that heat can play in the treatment of skin conditions such as acne.

Therefore, it is an object of the present invention to provide a device for treatment of acne. It is a further object to provide a hand-held device that treats acne by a combination of optical and thermal effects.

### SUMMARY OF THE INVENTION

The present invention is a hand-holdable device for treating acne at a treatment zone comprising a thermally conductive plate, a heater in thermal contact with the plate, one or more light emitting devices disposed to emit light of a first wavelength from the treatment device, one or more light emitting devices disposed to emit light of a second wavelength from the treatment device, and at least one switch that activates the heater and the light emitting devices upon contact between the plate and the treatment zone. The first wavelength is in the blue region of the spectrum and preferably in the range 400 nm to 410 nm. A first wavelength of 405 nm is particularly preferred. The second wavelength is in the red region of the spectrum and preferably in the range 630 nm to 640 nm. A second wavelength of 635 nm is particularly preferred.

Preferably, the device comprises a head portion and a handle portion, and the light emitting devices, thermally conductive plate, switch, and a power source are disposed within the head portion. Alternatively, the device comprises a body and a hand-holdable probe in which the light emitting devices, thermally conductive plate, and switch are disposed. Preferably, the thermally conductive plate is an aluminum plate. The switch is preferably a contact switch that closes a contact when pressure is applied by contacting the thermally conductive plate directly to the treatment zone.

### BRIEF DESCRIPTION OF THE DRAWINGS

FIG. 1 is a perspective view of a hand-held acne treatment device.

FIG. 2 is a top view of the acne treatment device of FIG. 1.

FIG. 3 is a cross-sectional right side view of the acne treatment device of FIG. 1, along the line 2-2 in FIG. 2;

FIG. 4 is a perspective view of an alternate embodiment of the hand-held acne treatment device.

FIG. 5 is a cross-sectional right side view of the acne treatment device of FIG. 1, along the line 2-2 in FIG. 2, showing the device pressed against a human face such that the switches contact the circuit board and activate the device.

FIG. 6 is a schematic view of an optical arrangement generating a linear spot shape of laser light.

FIG. 7 is a schematic view of an optical arrangement generating a circular spot shape of laser light.

FIG. 8 is an schematic illustration of the light emitting device configuration in the preferred embodiment of the present invention.

FIG. 9 is an schematic illustration of the light emitting device configuration in an alternate embodiment of the present invention.

### DETAILED DESCRIPTION OF THE INVENTION

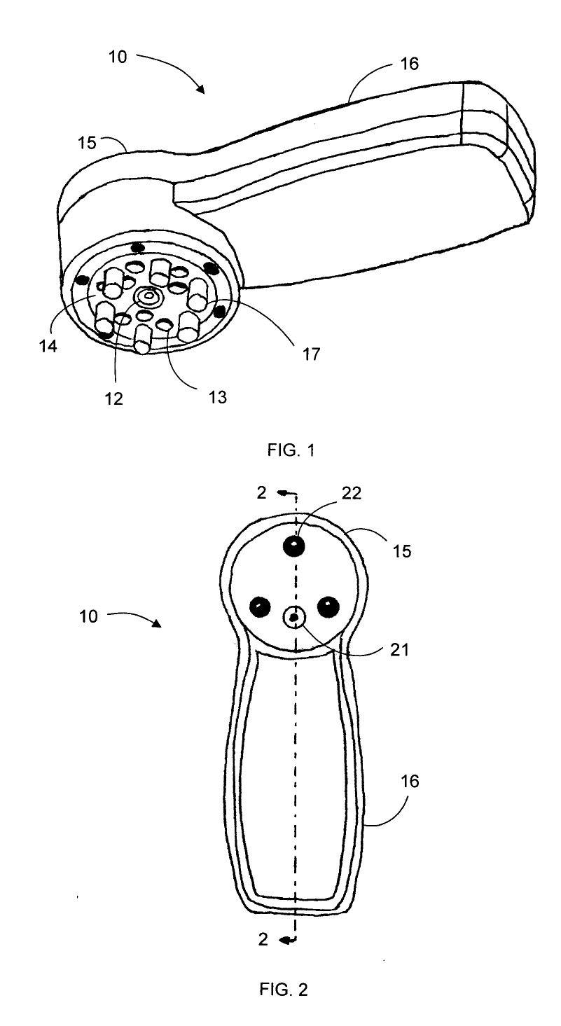

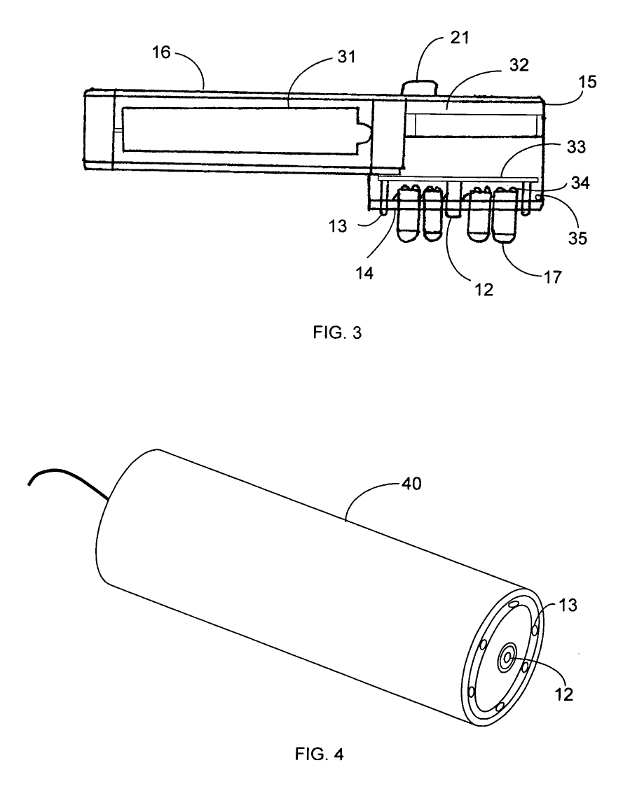

Referring to FIGS. 1-3 there is shown a hand-held acne treatment device 10 for treating acne in a treatment zone, typically the face, back, or chest. The device 10 is hand-holdable having a head portion 15 and a handle portion 16. The device 10 includes a first light emitting device 12 and one or more second light emitting devices 13. In an alternate embodiment, shown in FIG. 4, the device includes a handheld probe 40 in connection with a body (not shown). The probe 40 is preferably an elongated hollow tube defining an interior cavity which is shaped to be easily retained in a user's hand. The probe 40 may take on any shape that enables the laser light to be directed as needed such as tubular, T-shaped, substantially spherical, or rectangular (like a television remote control device). In this embodiment, the first and second light emitting devices 12, 13 are mounted in probe's 40 interior cavity, although the light emitting devices 12, 13 could be remotely located in the body and the laser light conducted by fiber optics to the probe 40. The components of the device that are not in the probe are disposed in the body.

The first and second light emitting devices 12, 13 may be semiconductor diodes, other laser light sources, light emitting diodes, or a combination thereof. In the preferred embodiment, the first light emitting device 12 is a laser light source, referred to herein as a laser, and the second light emitting devices 13 are light emitting diodes. The first light emitting device 12 and second light emitting devices 13 are mounted in, on, near or adjacent to a thermally conductive plate 14. As used hereinafter, “mounted in” comprises mounted in, on, near and adjacent to. The thermally conductive plate 14 is mounted in a head portion 15 of the acne treatment device 10 so as to be able to directly contact the treatment zone. Referring to FIG. 2, one or more indicator lights 22 indicate the operation of the device 10. For example, one indicator light 22 may indicate charging of the device, one may indicate operation of the heating element and another may indicate operation of the light emitting devices.

The first and second light emitting devices 12, 13 may be arranged in many different configurations, depending on the total number of light emitting devices mounted in the thermally conductive plate 14, the type or types of light emitting devices, whether each light emitting device emits light continuously or in pulses, and the wavelength of the light emitted by each light emitting device. In the preferred embodiment the first light emitting device 12 is situated in the center of the thermally conductive plate 14 and six second light emitting devices 13 are distributed around the thermally conductive plate 14. The first light emitting device 12 may operate at either red or blue wavelengths and the second light emitting devices 13 may operate at the same wavelength or a complementary wavelength. Preferably, the first light emitting device 12 operates at a red wavelength in the range of 630-640 nm, most preferably 635 nm, and the second light emitting devices 13 operate at a blue wavelength in the range of 400-410 nm, most preferably 405 nm.

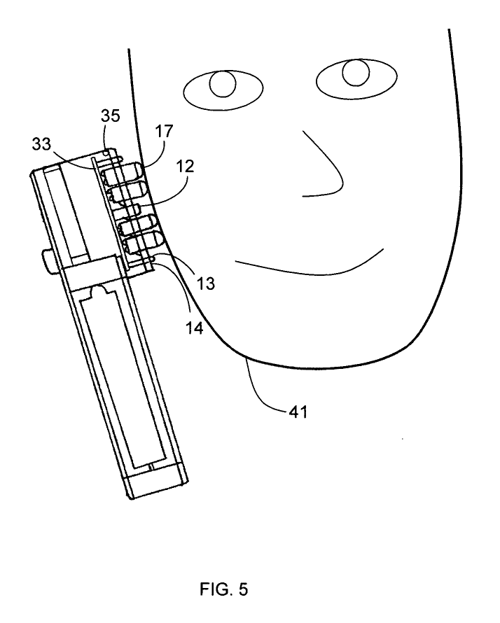

A user holds the treatment device 10 by a handle portion 16 and pushes the plate 14 against the treatment zone. In the alternate embodiment shown in FIG. 4, the user holds the probe 40 in his hand and pushes the plate 14 against the treatment zone. One or more contact sensors detect contact with the treatment zone and, after a predetermined period of time, activate the device. The predetermined period of time may be zero, such that contact with the plate and device activation are substantially simultaneous. For example, the contact sensors may be contact switches 17, which are biased away from a printed circuit board 33 by springs 34. When the user pushes the plate 14 against the treatment zone, such as the user's face 41, the switches 17 close a contact to activate the device 10, as shown in FIG. 5. Alternatively, the contact sensors may be proximity sensors that activate when the device 10 is held close to the treatment area. These proximity sensors may detect distance through acoustic, capacitive, inductive, infrared, or other means. Another alternative is to use a contact plate that activates when the electrical potential of the plate is dropped by contact with the lower potential treatment zone. Suitable circuits for these alternatives will be known to persons skilled in the art. The advantage of these alternatives is that the contact switches 17 are not required so a larger area of the plate 14 is available for applying treatment.

Upon activation, a heater 35 mounted in the plate 14 is energized to heat the plate 14, and hence heat the abutted treatment zone. The heater 35 may be any heating element capable of heating the plate 14 to a desired tempature and small enough to fit within the head 15, such as a resistive coil or a light emitting diode. In the preferred embodiment, the heater 35 is a resistive coil. A regulator on the printed circuit board 33 maintains the temperature of the plate 14 at a desired temperature. The first light emitting device 12 and second light emitting devices 13 are also energized and illuminate the treatment zone with a desirable fluence for a preset time and at a fixed wavelength. In a preferred method of operation there is a delay between the activation of the heater 35 and the activation of the first and second light emitting devices 12, 13. The delay allows the thermally conductive plate 14 to heat to an effective operating temperature, and thus dilate the pores, before application of the red and blue wavelengths.

The specific parameters of treatment time, fluence and wavelength are selected for the desired treatment modality and controlled by the circuit on the printed circuit board 33. One example is to use a first light emitting device 12 emitting in the range 630 nm to 640 nm with an irradiance of 1.8 mW/cm² for ten seconds and second light emitting devices 13 emitting in the range 400 nm to 410 nm with total irradiance of 0.9 mW/cm² for ten seconds. These parameters will result in a fluence of 18 mJ/cm² at the red wavelength and 9 mJ/cm² at the blue wavelength. The light emitting devices may be pulsed or continuous wave. If pulsed devices are used they are operated so that the total fluence is the same as a continuous wave embodiment.

The inventor speculates that the treatment at the blue wavelength targets naturally occurring porphyrin compounds, naturally occurring chromophores, and some naturally occurring acne bacteria. Other acne bacteria may be located deeper than the blue wavelength can adequately penetrate and these are treated by the red wavelength. In addition the red wavelength targets highly present macrophage cells to establish anti-inflammatory pathways. The heat from the thermally conductive plate 14 dilates the pores in the treatment zone and thus enhances the efficacy of the red and blue wavelengths.

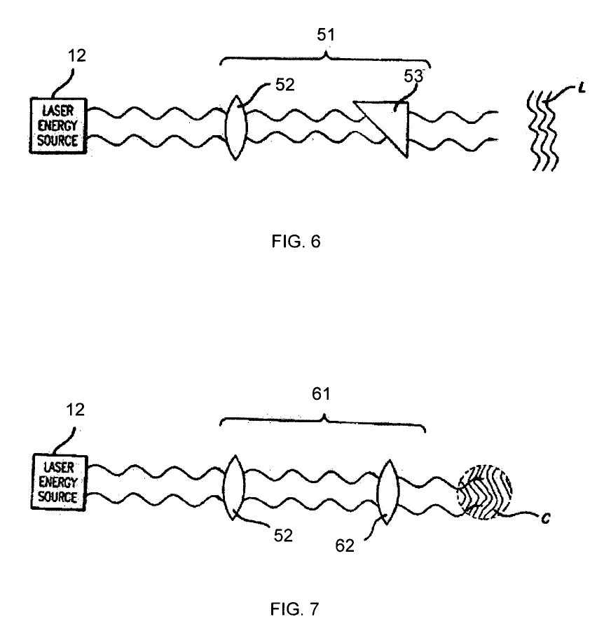

Light emitted from the light emitting devices may be shone through an optical arrangement that forms a beam spot in shapes that have therapeutic applications. The optical arrangement may comprise optical, electrical, mechanical, or software devices to shape the beam spot. The beam spot is the cross-sectional shape and size of the emitted beam as it exits the optical arrangement. For example, a laser beam of circular cross-section creates a circular beam spot as the laser light impinges the patient's skin. If the laser light emitted is in the visible range, a circular spot can be seen on the patient's skin of substantially the same diameter as the laser beam emitted from the optics arrangement. In one embodiment, shown in FIG. 6, emitted light is passed through a linear arrangement 51 that generates a beam of substantially linear cross-section, resulting in a line of laser light L seen on the patient's skin. The linear arrangement 51 includes a collimating lens 52 and a line generating prism 53. The collimating lens 52 and the line generating prism 53 are disposed in serial relation to, for example, the first light emitting device 12. The collimating lens 52 and the line generating prism 53 receive and transform the generated beam of laser light into the line of laser light L.

In another embodiment, shown in FIG. 7, emitted light passes through a circular arrangement that generates a beam of circular cross-section, resulting in a circular spot shape C as seen on the patient's skin. The circular arrangement 61 includes a collimating lens 52 and a second collimating lens 62. As with the first optical arrangement, the collimating lenses 52 and 62 are disposed in serial relation to, for example, the first light emitting device 12. The collimating lenses 52 and 62 receive and transform the generated beam of laser light into a circular beam spot of laser light C.

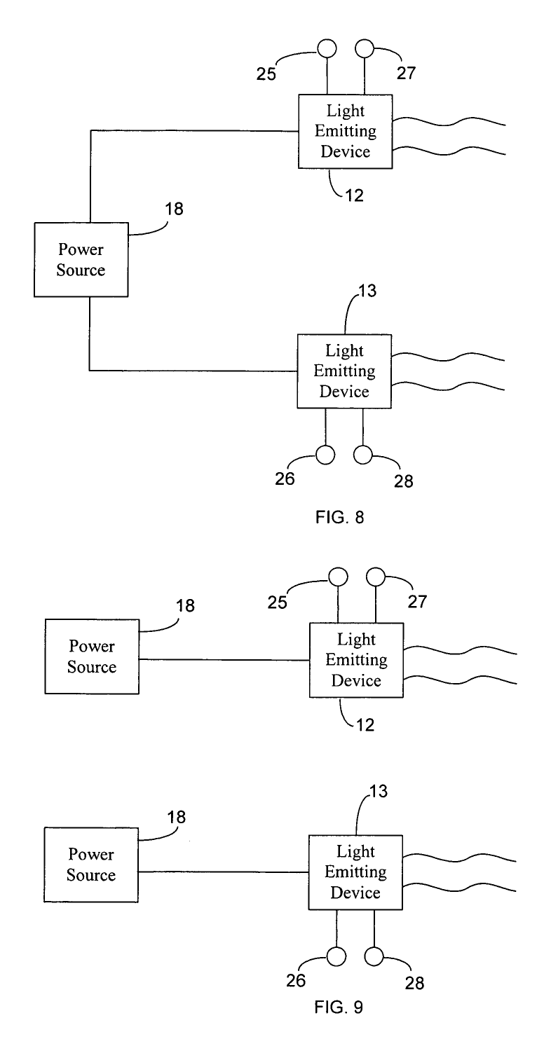

FIGS. 8 and 9 show the first light emitting device 12 and the second light emitting device 13 connected to a power source 18. The power source 18 may provide direct current, such as that provided by a battery, or alternating current, such as that provided by conventional building current, that is then converted to direct current. In the preferred embodiment, the first and second light emitting devices are connected to the same power source 18. In an alternate embodiment, the first and second light emitting devices are connected to different power sources 18. In the preferred embodiment, the power source 18 is one or more rechargeable batteries 31, which fit in the handle 16. A recharging circuit 32 in the head 15 receives mains power from charging port 21 to recharge the battery 31. A step down transformer (not shown) may provide power at a suitable voltage level to the charging port 21. Separate control circuits 25, 26 are connected to the light emitting devices 12, 13 respectively and act as on/off switches to control the period of time the laser light is generated. These laser energy sources can be energized independently or simultaneously which, throughout this specification, refers to acts occurring at generally at the same time. Control circuits 27, 28 are connected to the light emitting devices 12, 13, respectively, to form a control circuit that controls the duration of each pulse of laser light emitted, referred to herein as the pulse width. When there are no pulses, a continuous beam of laser light is generated. Pulse widths from 0 to 100,000 Hz may be employed to achieve the desired effect on the patient's tissue. Preferably, the treatment will deliver laser energy to the target tissue utilizing a pulse width short enough to sufficiently energize the targeted tissue and avoid thermal damage to adjacent tissue.

It will be understood that the device 10 provides a combination treatment of thermal and optical effects. The device may be employed by medical professionals and aestheticians. Additionally, the compact, hand-held device is easy for acne sufferers to use without seeking treatment of a medical professional. The contact switch or contact plate arrangement provides built-in safety and power conservation since the device only activates when the thermally conductive plate is in contact with the treatment zone. The voltages employed are low, the temperature is regulated and the fluences are at a safe level. While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A treatment device for treating a treatment zone, the device comprising:
a) a thermally conductive plate;
b) a heater in thermal contact with the thermally conductive plate;
c) at least one light emitting device that emits light of a first wavelength from the treatment device;
d) at least one light emitting device that emits light of a second wavelength from the treatment device; and
e) at least one contact sensor that activates the heater upon contact between the contact sensor and the treatment zone.

2. The device of claim 1 wherein the first wavelength is about 635 nm and the second wavelength is about 405 nm.

3. The device of claim 2 wherein the light of the first wavelength is on substantially simultaneously as the light of the second wavelength.

4. The device of claim 1 wherein:
a) at least one light emitting device that emits light of a first wavelength is a semiconductor diode; and
b) at least one light emitting device that emits light of a second wavelength is a light emitting diode.

5. The device of claim 1 further comprising a head portion wherein the light emitting devices are housed, the head portion attached to the thermally conductive plate.

6. The device of claim 1 further comprising a probe from which the light of the first and second wavelengths are emitted, the probe housing the light emitting devices within.

7. The device of claim 1 wherein the thermally conductive plate is an aluminum plate.

8. The device of claim 1 further comprising:
a) an optical arrangement for transforming light of the first wavelength into a desired beam spot shape.

9. The device of claim 8 wherein the desired beam spot shape is linear.

10. The device of claim 8 wherein the desired beam spot shape is circular.

11. The device of claim 1 wherein the heater is a resistive coil.

12. The device of claim 1 wherein the heater is a light emitting diode.

13. The device of claim 1 wherein, upon contact between the contact sensor and the treatment zone, the contact sensor activates at least one light emitting device that emits light of a first wavelength and at least one light emitting device that emits light of a second wavelength.

14. The device of claim 1 wherein, a predetermined time after contact between the contact sensor and the treatment zone, the contact sensor activates at least one light emitting device that emits light of a first wavelength and at least one light emitting device that emits light of a second wavelength.

15. The device of claim 14 wherein the predetermined time is zero.

16. The device of claim 1 wherein the contact sensor is a contact switch.

17. The device of claim 1 wherein the contact sensor is a contact plate.

18. The device of claim 1 wherein the contact sensor is a proximity sensor.

19. The device of claim 1 further comprising a battery attached to the contact sensor.

20. A treatment device for treating a treatment zone, the device comprising:
a) a thermally conductive plate;
b) a resistive coil in thermal contact with the thermally conductive plate;
c) one semiconductor diode that emits light of about 635 nm disposed at about the center of the thermally conductive plate;
d) at least three light emitting diodes that emit light of about 405 nm disposed around the periphery of the thermally conductive plate; and
e) at least one spring-loaded contact switch connected to each of the light emitting diodes that activate the resistive coil upon contact between the light emitting diodes and a patient's skin;
f) wherein components a-e are housed in a hand-held probe.

21. The treatment device of claim 20 further comprising a control circuit connected to the semiconductor diode and to the light emitting diodes.
